(19) United States
(12) Patent Application Publication
(10) Pub. No.: US 2018/0111745 A1
(43) Pub. Date: Apr. 26, 2018

(54) Glove Dispensing Assembly

(71) Applicant: Avent, Inc., Alpharetta, GA (US)

(72) Inventors: Shantilal Hirjibhai Modha, Milton, GA (US); Tantima Saelim, Songkhla (TH); Khoa Tuan Lien, Alpharetta, GA (US)

(73) Assignee: Avent, Inc., Alpharetta, GA (US)

(21) Appl. No.: 15/570,052

(22) PCT Filed: Apr. 7, 2016
(86) PCT No.: PCT/US16/26367

§ 371 Date: Oct. 27, 2017

Related U.S. Application Data
(60) Provisional application No. 62/251,371, filed on Nov. 5, 2015.
(60) Provisional application No. 62/155,238, filed on Apr. 30, 2015.

(51) Int. Cl. B65D 83/08; B65D 85/18; B65D 5/54; B65D 5/72

(52) U.S. Cl. CPC B65D 83/0817; B65D 85/18; A61B 42/40; B65D 5/724; B65D 5/542

(57) ABSTRACT

The present disclosure is directed to a dispensing assembly for dispensing articles. The dispensing assembly includes a container having a plurality of exterior panels that define an internal chamber. Further, the exterior panels include a top panel having an opening configured to dispense the articles therethrough. The dispensing assembly also includes an article dispensing component housed within the internal chamber. The article dispensing component includes a panel assembly arranged with the top panel of the container to form an article storage area. The panel assembly includes a first panel and a second panel secured to the first panel such that the second panel abuts against a lower side of the first panel. Further, the first panel defines a platform opposite the lower side configured to receive the plurality of articles. In addition, the second panel biases the first panel towards the top panel of the container.

### RELATED APPLICATIONS

The present application claims priority to U.S. Provisional Application Ser. No. 62/155,238 filed on Apr. 30, 2015 and U.S. Provisional Application Ser. No. 62/251,371 filed on Nov. 5, 2015 and International Application Number PCT/US2016/026367 filed on Apr. 7, 2016, which are incorporated herein in their entirety by reference hereto.

### FIELD OF THE INVENTION

The present invention relates in general to a dispensing assembly, and more particularly to a glove dispensing assembly and methods of manufacturing same.

### BACKGROUND OF THE INVENTION

A variety of single use, disposable products such as gloves, facemasks and the like are packaged in dispensing cartons. These dispensing cartons frequently have an opening or dispensing orifice cover.

Exemplary cartons or carton modifications for dispensing a variety of products including surgical gloves, tissues, dust mitts, and disposable gloves, are described at, for example:

U.S. Pat. No. 3,746,152 for “Surgical Glove Carton” issued to Allen on Jul. 17, 1973, describes a flat, sterilizable carton that is configured to store a pair of surgical gloves for an extended period in sterile condition and then “snap open” to a flat configuration and also lock in the flat configuration to present the surgical gloves to a user on a sterile field of cardboard.

U.S. Pat. No. 6,112,936 for “Medical Glove Dispensing Enclosure” issued to Arizmendi on Sep. 5, 2000, describes an envelope made of tubular net material that is stretched around a glove dispensing box or the like such that a sphincter closure in the net material is located over an opening in the dispensing box. The sphincter closure is made by cutting a hole in the net material and weaving an elastic band around the hole.

U.S. Pat. No. 6,488,175 for “Dusting Mitt Dispensing System” issued to Shiffler et al., on Dec. 3, 2002, describes a dispensing system for dispensing dusting mitts that have a thumb that is folded and arranged to present the thumb at the top of a stack of mitts. The dispensing system includes a carton having a top cover that pivots along a top seam at the back of the carton. The top cover contains a perforation pattern that forms an access flap when the perforations are severed. The access flap is integrally connected to the top cover along a seam to provide access to the contents.

U.S. Pat. No. 6,886,714 for “Container Allowing Choice of Multiple Openings for Dispensing Preference” issued to Kruchoski et al., on May 3, 2005, describes a dispensing container for dispensing sheets in which the container has a first dispensing opening for pop-up dispensing of sheets such as facial tissue. The container has a second opening to provide group dispensing of a plurality of sheets simultaneously without having to disassociate the sheets from each other. The openings can be overlaid on each other, or one opening can be placed on one portion of the container and another opening on another portion of the container to provide a consumer with various dispensing options.

U.S. Patent Application Publication No.: 2007/0210096 A1for “High-Volume Package Dispense” by Ellswood et al., published on May 3, 2005, describes a dispenser package system for protective articles, having a substantially vertical product storage orientation and package design. The package includes a double or multi-chambered dispenser unit that can provide within substantially the same footprint as a conventional dispenser container a greater volume of product. The package can store and dispense at least 50 percent, up to about 200 percent or greater capacity than conventional dispenser for protective article products such as gloves or face masks.

Thus, the art is continuously seeking new and improved dispensing assemblies for dispensing a variety of products. More specifically, assemblies that effectively dispense individual products, e.g. examination gloves, as the quantity of products become depleted to a level that the products are no longer located near the dispenser opening would be welcomed in the art. The need for such assemblies is particularly apparent for larger volume packages having contents that may settle or become difficult to access, particularly in a health care environment.

### BRIEF SUMMARY OF THE INVENTION

Aspects and advantages of the invention will be set forth in part in the following description, or may be obvious from the description, or may be learned through practice of the invention.

In one aspect, the present disclosure is directed to a dispensing assembly for dispensing articles. For example, the articles as described herein may include gloves, facemasks, paper products, dust mitts, or any other articles that would benefit from the dispensing assembly as described herein. The dispensing assembly includes a container having a plurality of exterior panels that define an internal chamber. Further, the exterior panels include a top panel having an opening configured to dispense the articles therethrough. The dispensing assembly also includes an article dispensing component housed within the internal chamber. The article dispensing component includes a panel assembly arranged with the top panel of the container to form an article storage area. The panel assembly includes a first panel and a second panel secured to the first panel such that the second panel abuts against a lower side of the first panel. Further, the first panel defines a platform opposite the lower side configured to receive the plurality of articles. In addition, the second panel biases the first panel towards the top panel of the container. Thus, the second panel is configured to move the first panel toward to the top panel as each article is dispensed from the opening.

In one embodiment, the first panel includes one or more side edges configured to abut against an internal wall of the container. Thus, the side edges provide stability to the first panel as it is being moved towards the top panel of the container via the second panel. In addition, the second panel may bias the first panel towards the top panel of the container via at least one flexible member. More specifically, in certain embodiments, the second panel may include a two-fold configuration having a first seam and a second seam. Thus, the first and second seams divide the second panel into a plurality of sections. More specifically, in certain embodiments, the plurality of sections may include a middle section and two opposing side sections. As such, the middle section of the second panel may be secured to the first panel, whereas the opposing side sections may be detached or free from the first panel. For example, in particular embodiments, the middle section of the second panel may be secured to the first panel via at least one of an adhesive or tape.

In further embodiments, the opposing side sections may be biased towards each other via the flexible member, e.g. an elastic or rubber band. In additional embodiments, each of the opposing side sections may include one or more notches configured to receive the flexible member. More specifically, in certain embodiments, each of the opposing side sections may include mirrored notches configured to receive the flexible member. In such an embodiment, the flexible member may be configured within the mirrored notches of each of the side opposing sections such that the flexible member biases opposing edges of the opposing sections towards each other. In addition, the mirrored notches may have any suitable cross-sectional shape, including for example an L-shaped cross-section.

In yet another embodiment, the article storage area may have open sides. In additional embodiments, the exterior panels of the container may include, in addition to the top panel, a bottom panel, opposing side panels and opposing end panels. Thus, at least one of the side panels or the end panels is configured to open to allow insertion of the article dispensing component. In addition, the articles as described herein may include any suitable article, including but not limited to gloves, facemasks, paper products, dust mitts, or similar.

In additional embodiments, the present disclosure is directed to package of articles. More specifically, the package of articles includes a container according to any of the preceding claims and a plurality of articles disposed within the article storage area of the container, wherein the plurality of articles is biased toward the top panel of the container as articles are dispensed from the opening.

In another aspect, the present disclosure is directed to a method for manufacturing a dispensing assembly. The method includes providing a first panel and a second panel. The method also includes forming a plurality of seams in the second panel so to create a middle section and two opposing side sections. The method also includes securing the middle section of the second panel to a lower side of the first panel as to form a panel assembly. Further, the method includes securing a flexible member to the opposing side sections of the second panel such that edges of the opposing side sections are biased towards each other. With the second panel being held flat, the method includes inserting the panel assembly into a container constructed of a plurality of exterior panels. The plurality of exterior panels may include at least a top panel having an opening configured to dispense a plurality of articles therethrough. Thus, another step includes placing a plurality of articles atop the first panel. Accordingly, as articles are dispensed from the opening, the flexible member biases the opposing side sections of the second panel towards each other so as to lift the first panel of the panel assembly towards the top panel of the container.

In one embodiment, the step of securing the middle section of the second panel to the lower side of the first panel as to form the panel assembly may further include bonding the middle section of the second panel to the lower side of the first panel via at least one of an adhesive or tape.

In additional embodiments, the opposing side sections may be detached or free from the first panel. In addition, the method may include forming one or more notches in each of the opposing side sections, with the one or more notches being configured to receive the flexible member. More specifically, as mentioned, the notches may be mirrored notches configured to receive the flexible member therein. Thus, in certain embodiments, the flexible member may include an elastic band configured within the mirrored notches of each of the side opposing sections. It should also be understood that the dispensing assembly may be further configured with any one of or a combination of additional features as described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of one embodiment of a dispensing assembly for dispensing articles according to the present disclosure;

FIG. 2 illustrates a perspective view of one embodiment of a container of a dispensing assembly according to the present disclosure;

FIG. 3 illustrates a perspective view of one embodiment of a panel assembly of a dispensing assembly according to the present disclosure, particularly illustrating an unfolded second panel that is subsequently folded together via a flexible member;

FIG. 4 illustrates a perspective view of one embodiment of a second panel of a panel assembly of a dispensing assembly according to the present disclosure, particularly illustrating the second panel in a folded configuration;

FIG. 5 illustrates a perspective view of one embodiment of a second panel of a panel assembly of a dispensing assembly according to the present disclosure, particularly illustrating the second panel in an unfolded configuration;

FIG. 6 illustrates an internal, perspective view of one embodiment of dispensing assembly according to the present disclosure, particularly illustrating a panel assembly having a second panel that is biasing a first panel towards an opening of a container of the dispensing assembly;

FIG. 7 illustrates a perspective view of one embodiment of dispensing assembly according to the present disclosure, particularly illustrating a second panel secured to a first panel in an unfolded configuration;

FIG. 8 illustrates a perspective view of one embodiment of a panel assembly of a dispensing assembly according to the present disclosure, particularly illustrating a folded second panel and a first panel having folded side edges;

FIG. 9 illustrates a top view of one embodiment of a second panel of a panel assembly of a dispensing assembly according to the present disclosure, particularly illustrating the second panel in an unfolded configuration with each of the sections of the second panel having about the same length;

FIG. 10 illustrates a top view of one embodiment of a first panel of a panel assembly of a dispensing assembly according to the present disclosure;

FIG. 11 illustrates a perspective view of another embodiment of a panel assembly of a dispensing assembly according to the present disclosure, particularly illustrating a folded second panel and a first panel having rounded corners;

FIG. 12 illustrates a top view of another embodiment of a second panel of a panel assembly of a dispensing assembly according to the present disclosure, particularly illustrating the second panel in an unfolded configuration with the side sections of the second panel having a longer length than the middle section;

FIG. 13 illustrates a top view of another embodiment of a first panel of a panel assembly of a dispensing assembly according to the present disclosure;

FIG. 14 illustrates a flow diagram of one embodiment of a method of manufacturing a dispensing assembly according to the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Dispensing disposable examination gloves from a package can be particularly difficult. For smaller-sized examination gloves, such as, for example, standard small or extra-small sizes, users may wish to have a smaller orifice to access the contents of the carton to prevent gloves from spilling out. The size of these gloves may be close to or not much larger than the size of the orifice. In some cases, the dimensions of the gloves may be smaller than the size of the orifice. This is particularly notable when the dispensing carton is initially opened and the contents are immediately adjacent the orifice. However, for larger-sized examination gloves, such as, for example, standard large or extra-large sizes, users may wish to have a larger orifice to provide for easier dispensing from the carton. The size of these gloves may be larger or even much larger than a typical glove dispensing orifice.

In order to improve economy, dispensing assemblies or packages are frequently larger in size to hold larger quantities of articles. When dispensing articles such as, for example, disposable examination gloves from a larger carton or package, dispensing becomes problematic after a sufficient quantity of articles (e.g., gloves) is depleted since the gloves are no longer readily accessible near the dispensing opening. Users are forced to tip the dispensing assembly or package or even insert their fingers or entire hand deep into the package to grasp and withdraw an article. The opening is typically about the same size as the user's hand, so mobility as well as vision inside the package (e.g., the chamber of the dispensing assembly) is restricted.

Thus, the present invention is directed to an improved dispensing assembly that allows the gloves of the assembly to be pushed closer to the opening to enhance access to the interior of the container. This is particularly important for larger volume packages having contents that may settle or become difficult to access, especially in a health care environment.

For example, the present invention allows the user to remove a certain number of articles with each subsequent article being just as readily available as the previous article. More specifically, as the contents settle or a sufficient quantity of articles (e.g., gloves) is depleted such that the gloves are no longer readily accessible near the orifice, the present invention allows the first panel of the article dispensing component to be biased towards the top panel of the container such that the articles remain close to the dispensing opening. Thus, the dispensing assembly of the present disclosure is suitable for use with large containers.

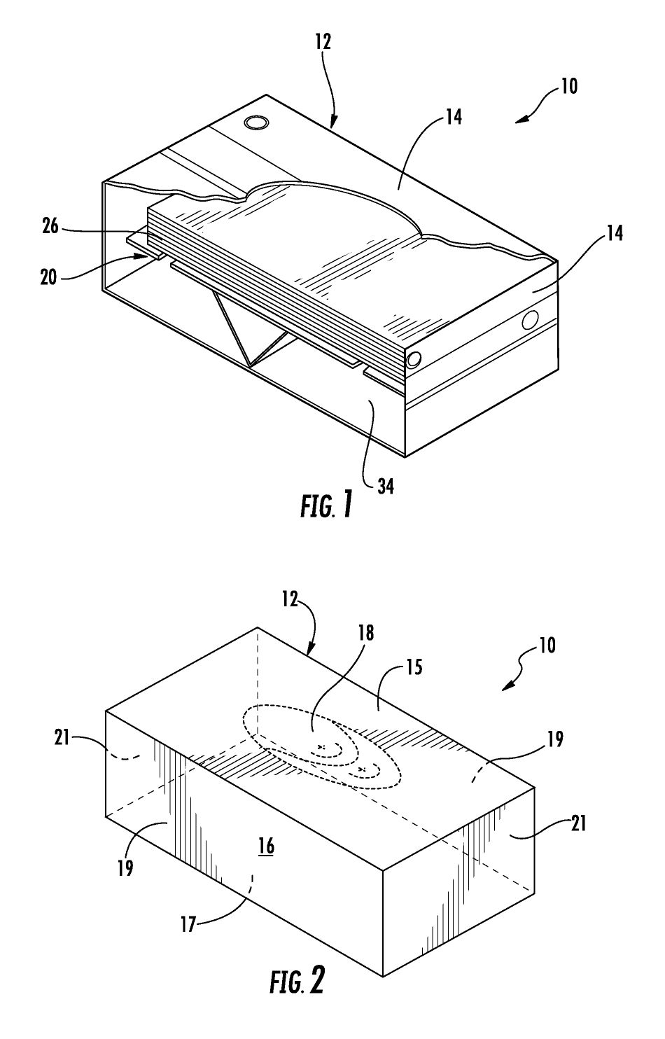

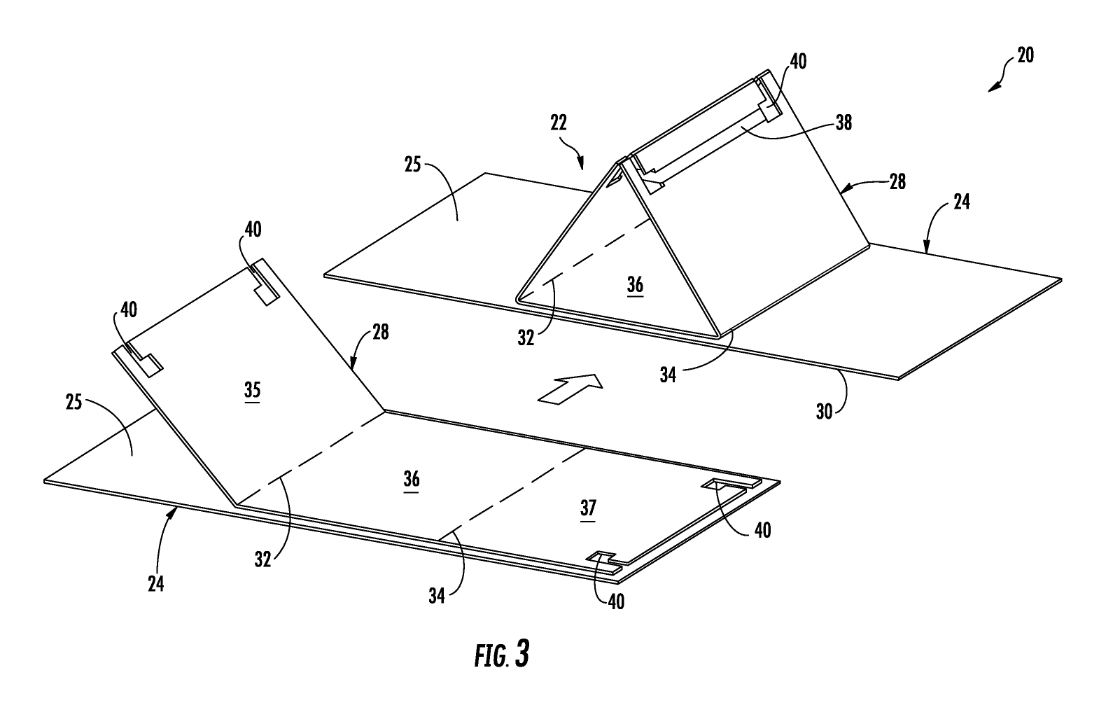

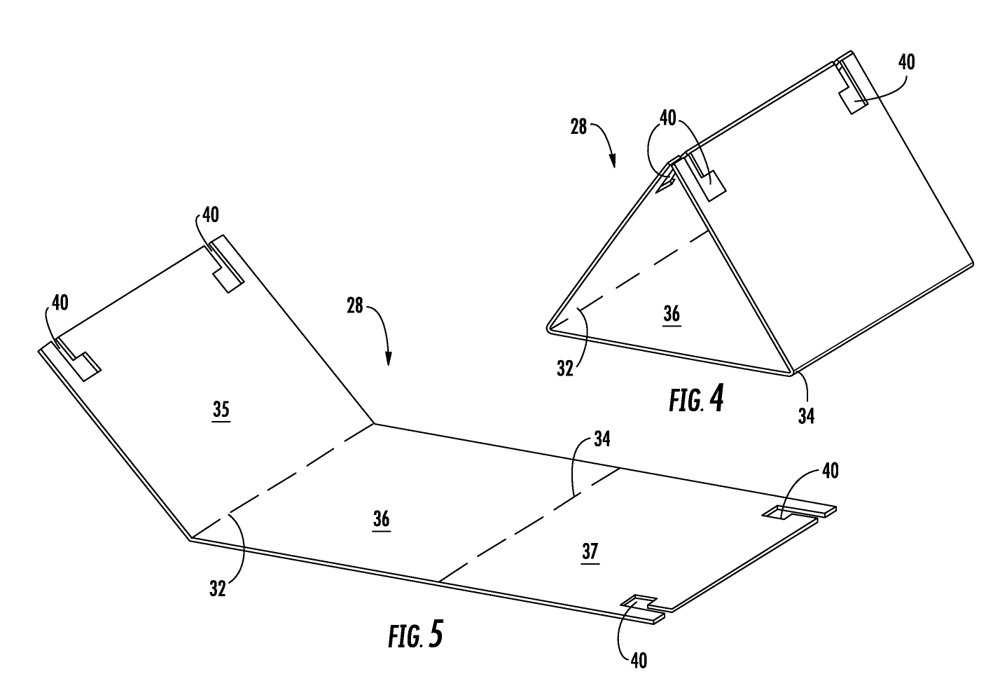

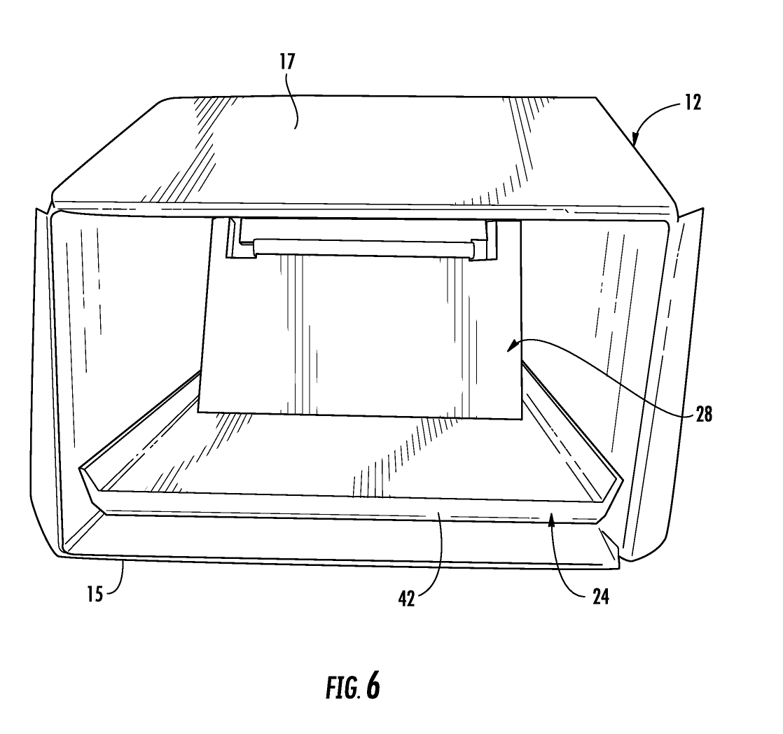

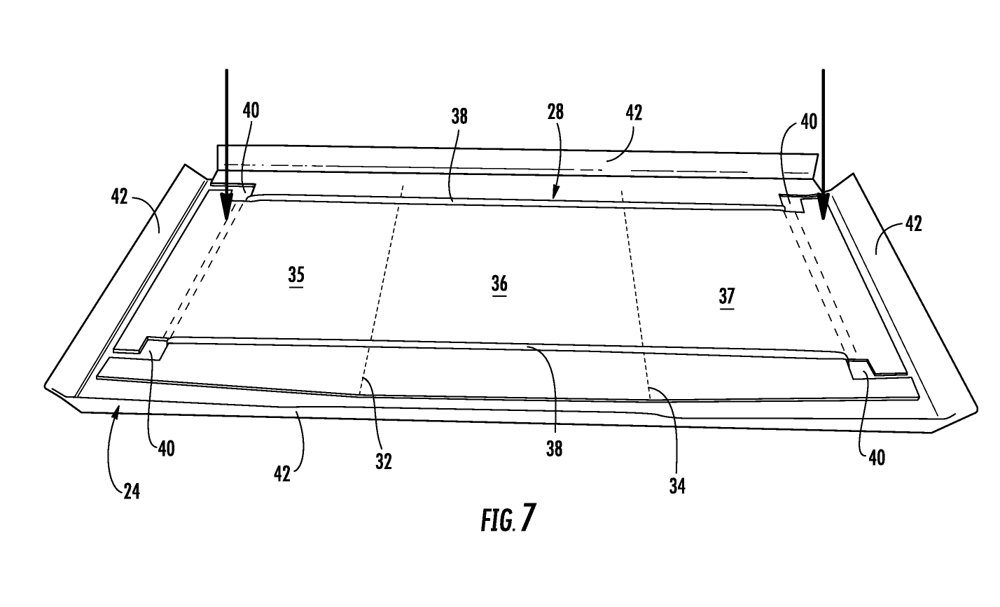

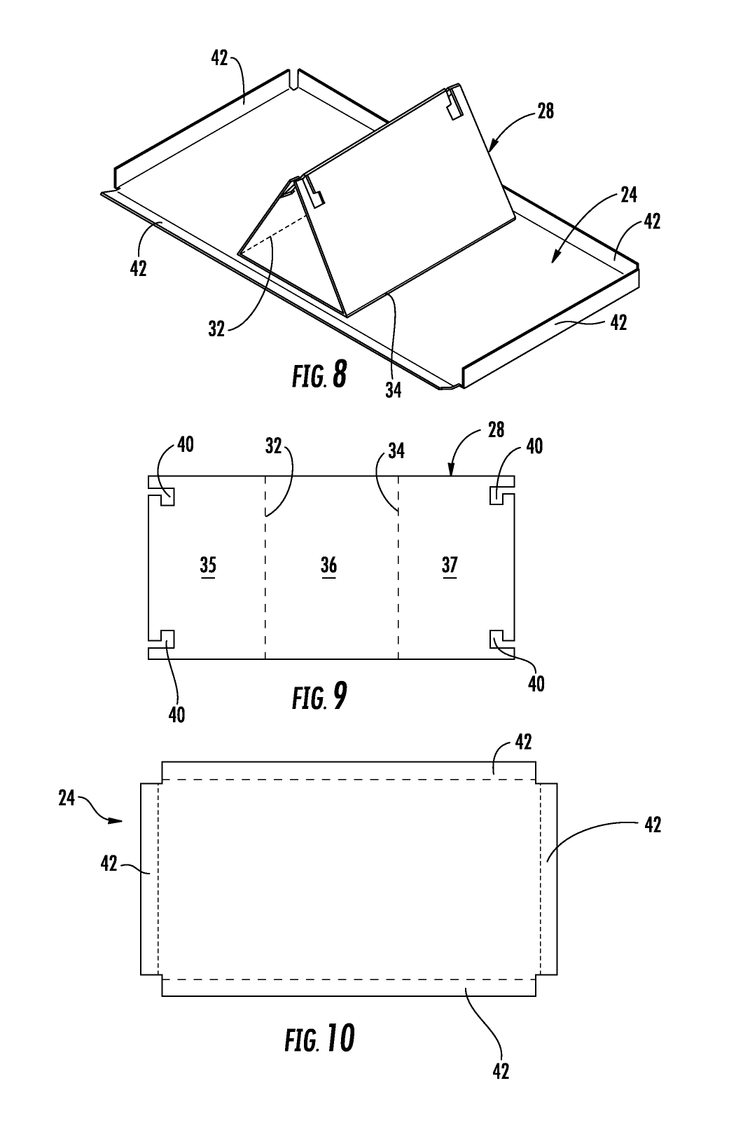

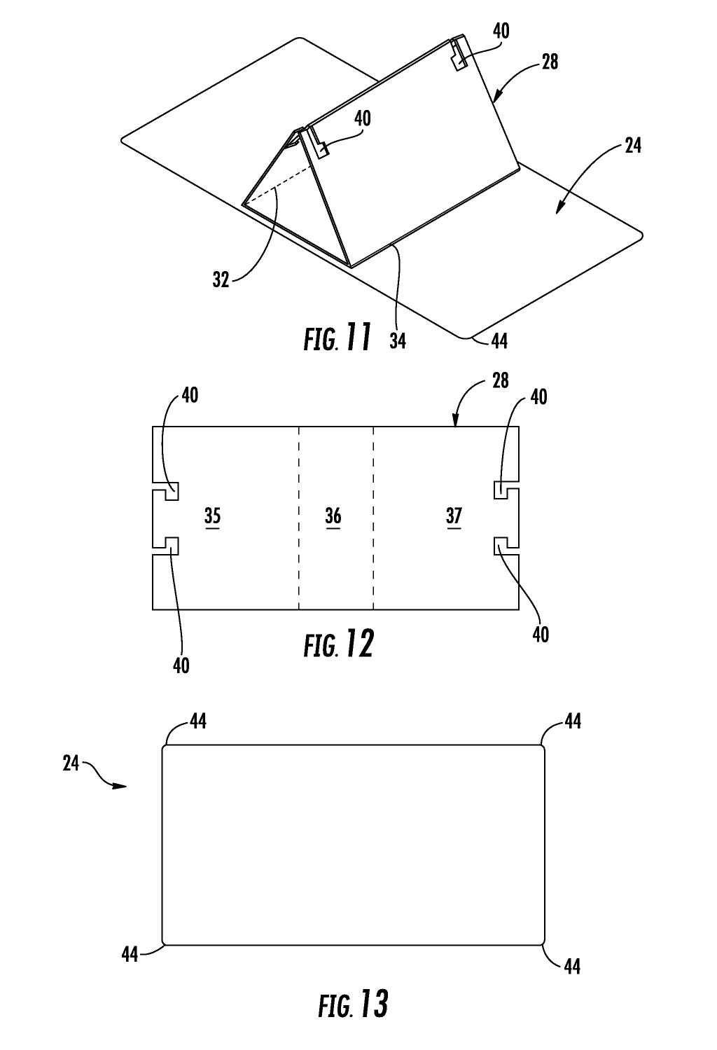

Referring now to the drawings, FIG. 1 illustrates a perspective, cut-away view of an exemplary dispensing assembly 10 or package for dispensing articles such as, for example, disposable examination gloves, facemasks, paper products (e.g. tissues, paper towels, etc.) dust mitts, or the like. As shown, the dispensing assembly 10 includes a container 12 having a plurality of exterior panels 14. The exterior panels 14 may be constructed of any suitable material such as, for example, carton cardboard stock, paperboard, heavy structural paper, container stock, corrugated paperboard, plastic coated paper, plastic sheets, wax-coated papers or the like, and combinations thereof. Further, as shown in FIG. 2, the exterior panels 14 of the container 12 are configured to define an internal chamber 16.

More specifically, as shown in FIG. 2, the exterior panels 14 include a top panel 15, a bottom panel 17, opposing side panels 19, and opposing end panels 21. Thus, in certain embodiments, either or both of the side panels 19 or the end panels 21 may be configured to open, thereby allowing insertion or removal of a plurality of articles as described herein. In addition, in certain embodiments, the exterior panels 14 may be integral with each other. Thus, in such embodiments as shown, the exterior panels 14 may be constructed of a unitary piece of material divided into multiple parts via one or more seams that can be easily folded along each seam to form the container 12. Alternatively, the exterior panels 14 may be separate and detached pieces of material e.g. that may be joined together via any suitable means, including but not limited to adhesive, tape, clamps, or similar. In further embodiments, certain panels of the container 12 may be excluded, for example, the side panels, or any other suitable combination.

Further, as shown generally in the figures, the top panel 15 includes an opening 18 configured to dispense a plurality of articles therethrough. In addition, as shown, the opening 18 has a generally oval shape, however, it should be understood that the opening 18 may have any other suitable shape such that one or more articles can be dispensed therethrough. Further, as shown in FIG. 2, the opening 18 may include a removable section defined by perforations, scores, underscores, or partial cuts through the material and combinations thereof. Such features are known to those of ordinary skill in the art. For example, U.S. Pat. No. 4,158,412 for “Tear Out Opening Device” issued to Wysocki on Jun. 19, 1979, describes half-cut configurations used for a tear out flap, the contents of which are incorporated herein by reference in its entirety. In addition, the opening 18 may be configured according to U.S. Pat. No. 8,646,653 entitled “Dispensing Assembly and Package of Articles” which is incorporated herein by reference in its entirety.

Referring particularly to FIGS. 1 and 3, the dispensing assembly 10 also includes an article dispensing component 20 housed within the internal chamber 16. More specifically, as shown, the article dispensing component 20 includes a panel assembly 22 arranged with the top panel 15 of the container 12 to form an article storage area 26. Thus, the article storage area 26 is configured to receive the plurality of articles therein. More specifically, as shown in the illustrated embodiment, the panel assembly 22 forms the bottom of the article storage area 26 and the top panel 15 of the container 12 forms the top of the article storage area 26. In additional embodiments, the article storage area 26 may have open sides.

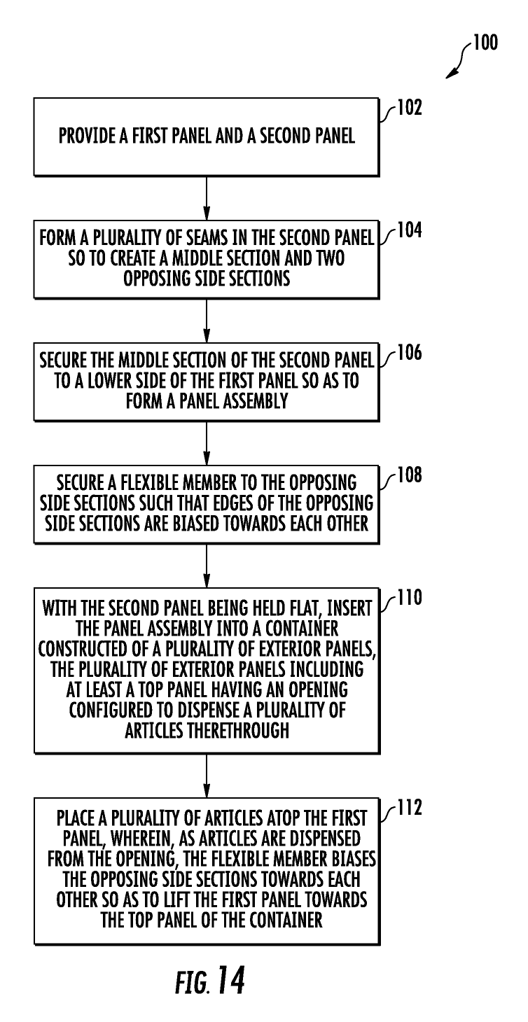

Referring generally to FIGS. 3-13, the panel assembly 22 also includes a first panel 24 and a second panel 28 secured to the first panel 24 such that the second panel 28 abuts against a lower side 30 of the first panel 24. Further, the first panel 24 defines a platform 25 opposite the lower side 30 configured to receive the plurality of articles. Thus, the second panel 28 is configured to bias the first panel 24 towards the top panel 15 of the container 12.

More specifically, as shown in the illustrated embodiment, the second panel 28 is configured to bias the first panel 24 towards the top panel 15 of the container 12 via at least one flexible member 38. For example, as shown, the top panel 15 of the container, by design, is stationary, whereas the first panel 24 is biased towards the top panel 15 via the second panel 28. In other words, the first panel 24 may be configured to move relative to the top panel 15 as each article (e.g. glove) is dispensed from the opening 18 of the container 12. For example, as shown in the figures, the second panel 28 may include a two-fold configuration having a first seam 32 and a second seam 34. Thus, the first and second seams 32, 34 are configured to divide the second panel 28 into a plurality of sections 35, 36, 37. More specifically, in certain embodiments, the plurality of sections 35, 36, 37 may include a middle section 36 and two opposing side sections 35, 37. As such, the middle section 36 of the second panel 28 may be secured to the first panel 24, whereas the opposing side sections 35, 37 may be detached or free from the first panel 24. For example, in particular embodiments, the middle section 36 of the second panel 28 may be secured to the first panel 24 via at least one of an adhesive or tape. In addition, as shown in FIG. 9, each of the sections 35, 36, 37 may have substantially the same length. Alternatively, as shown in FIG. 12, the side sections 35, 37 may be longer than the middle section 36. More specifically, the side sections 35, 37 of the second panel 28 may be longer than the height of the opposing end panels 19 and opposing side panels 21. This relationship between the length of the side sections 35, 37 and the panels 19, 21 assists in preventing one or both of the sides sections 35, 37 from folding under.

Referring to FIGS. 3, 6, and 7, the flexible member 38, e.g. an elastic or rubber band, is configured to bias the opposing side sections 35, 37 towards each other so as to lift the first panel 24 of the panel assembly 22 towards the top panel 15 of the container 12 as the articles are dispensed from the opening 18. More specifically, in certain embodiments, each of the opposing side sections 35, 37 may include one or more notches 40 configured to receive the flexible member 38. For example, as shown, each of the opposing side sections 35, 37 may include mirrored notches 40 configured to receive the flexible member 38. As used herein, the term “mirrored” generally encompasses notches having corresponding shapes that are oriented opposite of each other. In such an embodiment, the flexible member 38 may be configured within the mirrored notches 40 of each of the side opposing sections 35, 37 so as to bias the opposing sections 35, 37 towards each other. In addition, as shown, the mirrored notches 40 may have any suitable cross-sectional shape. For example, as shown, the mirrored notches 40 have an L-shaped cross-section. Further, as shown in FIG. 9, the notches 40 may be located adjacent to the corners of the second panel 28. Alternatively, as shown in FIG. 12, the notches may be spaced closer together so as to reduce the tension provided by the flexible member 38. Alternatively and/or additionally, the tension in the flexible member 38 may also be altered by changing the size/dimensions of the flexible member 38 and/or the material of the flexible member 38. By reducing the tension in the flexible member 38, the dispensing assembly can be more easily manufactured and assembled. In addition, the inventors of the present disclosure have discovered that the flexible member 38 does not have to provide tension to urge a full load of articles (e.g. gloves) towards the dispensing opening 18, but only needs to provide sufficient tension to urge less than a full load of articles towards the opening 18. Further, the required tension steadily decreases as the articles are dispensed from the opening 18.

In an aspect of the invention, the flexible member 38 is initially aligned or oriented in a direction that is generally parallel with the top and bottom panels 15, 17 of the container 12. This configuration helps save space and increases the volume available for a load of articles. Further, such a configuration also aids in the method of loading articles as described below. Moreover, this configuration allows a relatively high level of initial stretch or displacement of the flexible member 38 which provides the required levels of tension to urge a full load of articles towards the dispensing opening 18. However, the energy of the flexible member 38 (i.e., when under tension) is oriented in a direction that is generally parallel with the top and bottom panels 15, 17 of the container 12 and not advantageous or useful for urging a full load of articles towards the dispensing opening. Thus, the energy of the flexible member 38 must be reliably and robustly translated or converted in a perpendicular direction (e.g., toward the dispensing opening 18) without adding complexity, expense, and/or weight to the dispensing assembly 10. This is not a trivial problem to solve because the dispensing assembly 10 itself is generally designed as a single-use, disposable dispenser box that typically is used to dispense single-use, inexpensive articles such as, for example, medical exam-type gloves, face masks or the like. Importantly, the second panel 28 is configured to reliably and robustly translate or convert the energy of the flexible member 38 from a direction that is generally parallel with the top and bottom panels 15, 17 of the container 12 without adding complexity, expense, and/or weight to the dispensing assembly 10.

It should be understood that the flexible members 38 as described herein may include any suitable members configured to bias the first panel 24 towards the top panel 15 of the container 12, e.g. elastic or rubber bands, springs, etc. More specifically, as shown, the flexible members 38 may be elastic bands. Accordingly, the flexible members 38 are configured to allow movement of the first panel 24 toward to the top panel 15 as each article is dispensed from the opening 18. In addition, any number of flexible members 38 may be used. For example, as shown generally in the figures, the second panel 28 includes one flexible member 38. In additional embodiments, the article dispensing component 20 may include more than one flexible member 38.

In additional embodiments, as shown in FIGS. 6-8, 10, the first panel 24 may also include one or more side edges 42 configured to abut against an internal wall of the container 12. Thus, in such embodiments, the side edges 42 are configured to move along the internal wall of the container 12 as the first panel 24 is biased towards the top panel 15 of the container 12 by the folding second panel 28, thereby providing increased stability to the first panel 24 as it is moved via the second panel 28. In alternative embodiments, as shown in FIGS. 11-13, the side edges 42 may be omitted and the first panel 24 may simply include rounded corners 44. Thus, in such embodiments, the rounded corners 44 are configured to reduce pinching of the articles, thereby reducing the articles from becoming stuck.

Referring now to FIG. 14, a flow diagram of one embodiment of a method 100 for manufacturing a dispensing assembly 10 for dispensing articles such as gloves, facemasks, paper products, dust mitts, or the like according to the present disclosure is illustrated. As shown at 102, the method 100 includes providing a first panel 24 and a second panel 28. As shown at 104, the method 100 includes forming a plurality of seams (e.g. seams 32 and 34) in the second panel 28 so to create a middle section 36 and two opposing side sections 35, 37.

As shown at 108, the method 100 includes securing the middle section 36 of the second panel 28 to the lower side 30 of the first panel 24 as to form the panel assembly 22. More specifically, in certain embodiments, the step of securing the middle section 36 of the second panel 28 to the lower side 30 of the first panel 24 as to form the panel assembly 22 may further include bonding the middle section 36 of the second panel 28 to the lower side 30 of the first panel 24 via at least one of an adhesive or tape.

As shown at 110, the method 100 also includes securing a flexible member 38 to the opposing side sections 35, 37 such that edges of the opposing side sections 35, 37 are biased towards each other (FIG. 3). With the second panel 28 being held flat (FIG. 7), as shown at 112, the method 100 includes inserting the panel assembly 22 into the container 12. As shown at 114, the method 100 includes placing a plurality of articles atop the first panel 24. Accordingly, as articles are dispensed from the opening 18 of the container 12, the flexible member 38 biases the opposing side sections 35, 37 towards each other so as to lift the panel assembly 22 towards the top panel 15 of the container 12.

In additional embodiments, the opposing side sections 35, 37 may be detached or free from the first panel 24. In addition, the method 100 may include forming one or more notches 40 in each of the opposing side sections 35, 37, with the one or more notches 40 being configured to receive the flexible member 38. More specifically, as mentioned, the notches 40 may be mirrored notches configured to receive the flexible member 38. Thus, in certain embodiments, the flexible member 38 may include an elastic band configured within the mirrored notches 40 of each of the side opposing sections 35, 37.

The plurality of articles as described herein is desirably a plurality of disposable articles. As used herein, the term “disposable” refers to a product that is so inexpensive that it may economically be discarded after only a single use. Products that are “disposable” are typically intended for single use. The term “single-use” refers to a product that is intended to be used only once and is not intended to be re-used, re-conditioned, restored or repaired after that use. Such products offer advantages in clinical settings by reducing the potential for contamination or infection. In addition, these products can enhance work flow since they are not collected and assembled for reprocessing and reuse. Examples of disposable articles include disposable examination gloves, disposable facemasks and the like.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

1. A dispensing assembly, comprising:
a container comprising a plurality of exterior panels defining an internal chamber, the plurality of exterior panels comprising at least a top panel having an opening configured to dispense a plurality of articles therethrough; and
an article dispensing component housed within the internal chamber, the article dispensing component comprising a panel assembly arranged with the top panel of the container to form an article storage area, the panel assembly comprising a first panel and a second panel secured to the first panel such that the second panel abuts against a lower side of the first panel, the first panel defining a platform opposite the lower side configured to receive the plurality of articles,
wherein the second panel biases the first panel towards the top panel of the container as each article is dispensed from the opening.

2. The dispensing assembly of claim 1, wherein the first panel comprises one or more side edges configured to abut against an internal wall of the container.

3. The dispensing assembly of claim 1, wherein the first panel comprises one or more rounded corners.

4. The dispensing assembly of claim 1, wherein the second panel biases the first panel towards the top panel of the container via at least one flexible member.

5. The dispensing assembly of claim 1, wherein the second panel comprises a two-fold configuration comprising a first seam and a second seam, the first and second seams dividing the second panel into a plurality of sections.

6. The dispensing assembly of claim 4, wherein the plurality of sections comprises a middle section and two opposing side sections.

7. The dispensing assembly of claim 1, wherein the exterior panels of the container further comprise a bottom panel, opposing side panels and opposing end panels, wherein at least one of the side panels or the end panels are configured to open to allow insertion of the article dispensing component.

8. The dispensing assembly of claim 7, wherein the two opposing side sections are longer than a height of the opposing end panels and the opposing side panels.

9. The dispensing assembly of claim 6, wherein the middle section of the second panel is secured to the first panel and the opposing side sections are detached from the first panel.

10. The dispensing assembly of claim 9, wherein the middle section of the second panel is secured to the first panel via at least one of an adhesive or tape.

11. The dispensing assembly of claim 9, wherein edges of the opposing side sections are biased towards each other via the flexible member.

12. The dispensing assembly of claim 11, wherein each of the opposing side sections comprises one or more notches configured to receive the at least one flexible member.

13. The dispensing assembly of claim 12, wherein each of the opposing side sections comprises mirrored notches configured to receive the at least one flexible member.

14. The dispensing assembly of claim 13, wherein the mirrored notches comprise an L-shaped cross-section.

15. The dispensing assembly of claim 1, wherein the article storage area comprises open sides.

16. The dispensing assembly of claim 1, wherein the articles further comprise at least one of gloves, facemasks, paper products, or dust mitts.

17. A package of articles comprising:
a container according to claim 1; and
a plurality of articles disposed within the article storage area,
wherein the plurality of articles is biased toward the top panel of the container as articles are dispensed from the opening.

18. A method for manufacturing a dispensing assembly, the method comprising:
providing a first panel and a second panel;
forming a plurality of seams in the second panel so to create a middle section and two opposing side sections;
securing the middle section of the second panel to a lower side of the first panel as to form a panel assembly;
securing a flexible member to the opposing side sections such that edges of the opposing side sections are biased towards each other;
with the second panel being held flat, inserting the panel assembly into a container constructed of a plurality of exterior panels, the plurality of exterior panels comprising at least a top panel having an opening configured to dispense a plurality of articles therethrough; and,
placing a plurality of articles atop the first panel,
wherein, as articles are dispensed from the opening, the flexible member biases the opposing side sections towards each other so as to lift the panel assembly towards the top panel of the container.

19. The method of claim 18, wherein securing the middle section of the second panel to the lower side of the first panel as to form a panel assembly further comprises bonding the middle section of the second panel to the lower side of the first panel via at least one of an adhesive or tape.

20. (canceled)

21. The method of claim 18, further comprising forming one or more notches in each of the opposing side sections, the one or more notches configured to receive the flexible member.

22-25. (canceled)
